(19) United States
(12) Patent Application Publication
(10) Pub. No.: US 2014/0275976 A1
(43) Pub. Date: Sep. 18, 2014

(54) Global Ventricular Cardiac Diastolic Function Evaluation System and Associated Methods

(71) Applicant: ADVENTIST HEALTH SYSTEM/SUNBELT, INC., Altamonte Springs, FL (US)

(72) Inventor: Richard J. Moro, Winter Park, FL (US)

(73) Assignee: ADVENTIST HEALTH SYSTEM/SUNBELT, INC., Altamonte Springs, FL (US)

(21) Appl. No.: 14/108,803

(22) Filed: Dec. 17, 2013

Related U.S. Application Data
(60) Provisional application No. 61/790,296, filed on Mar. 15, 2013.

(51) Int. Cl. A61B 5/029; A61B 5/024; A61B 6/00; A61B 6/03; A61B 8/08; A61B 5/00; A61B 5/026

(52) U.S. Cl. CPC A61B 5/029; A61B 5/7275; A61B 5/02405; A61B 5/7282; A61B 5/0263; A61B 6/032; A61B 8/0883; A61B 6/503
USPC 600/419; 600/526; 600/425; 600/450; 600/436

(57) ABSTRACT

A method for evaluating diastolic function of a heart includes measuring a volumetric flow of blood through the heart and determining volume change rates during a diastolic flow period. A diastolic index is formulated from a combination of volume change rates and features of the volumetric change and is weighted by the index for evaluating the weighted feature at a heightened sensitivity against a preselected value. The index weighting provides a measure of diastolic filling performance specific to the weighting parameter. As a result, guidance is provided in evaluating volume changes in heart failure patients, cardiac diastolic performance, medication/titration for diastolic performance, an athletic training program, and cardiac reserve. Guidance is also provided for improving exercise capacity in patients with diastolic dysfunction without requiring the patient to be evaluated during exertion.

### CROSS-REFERENCE TO RELATED APPLICATION

This application claims the benefit of U.S. Provisional Application No. 61/790,296, having filing date of Mar. 15, 2013 the disclosure of which is hereby incorporated by reference in its entirety and all commonly owned.

### FIELD OF THE INVENTION

Embodiments of the invention generally relate to the field of cardiac function determination, and more specifically to systems and methods for evaluating global right and left ventricular cardiac diastolic function and improving diagnosis and treatment of diastolic dysfunction and diastolic heart failure.

### BACKGROUND

Failure to properly fill the ventricle during diastole is known as diastolic dysfunction (DD) or diastolic heart failure (DHF). Recent studies have demonstrated that in addition to DD and DHF, systolic heart failure (SHF) may occur individually, and clinical manifestations of both DHF and SHF may be present concurrently in some. Consequently, patients can have either a dilated LV with a poor ejection fraction, SHF, or a normal-size LV having a normal ejection fraction percent (% EF) experiencing DHF during activity, thereby decreasing the patient's quality of life. Treatment of SHF and DHF often diverge, but the clinical presentations are similar making it difficult to differentiate. Therefore, it would be beneficial to provide a system and method that improves the measurement of a patient's diastolic filling dynamics and their proper diagnostic classification for treatment and care.

Cardiac ventricular functional analysis is a key component in determining the significance of cardiac disease. The majority of effort and data is directed to ventricular systolic function providing very defined outputs such as EF %, dP/dt, and SV. The ability of the heart to provide sufficient output at any given state is most often dependent on the management of the diastolic filling period and always dependent on diastolic filling dynamics. There has been a need for a method of quantifying volumetric relationships of diastolic filing. Embodiments of the present invention utilize a diastolic index (herein referred to as a Moro Index™) and provide methods of quantifying volumetric relationships of diastolic filling using a performance index, herein referred to as a weighted Moro Index including a weighting of the Moro Index™ (MI) with a volumetric feature, by way of example, and referred to as a MI_{feature}.

Diastolic function is an important component of cardiac output contributing to the heart failure (HF) syndrome in 60-70% of patients afflicted. Diastolic function and DHF remain difficult to quantify. Current solutions allow a simple comparison determination of “better or worse” and are restricted in scope often offering a simple snapshot of the diastolic or filling phase of the LV. Also, these solutions evaluate flow velocity directly or indirectly measure rates of volume change. Each of these known methods can be affected by structural changes such as atrial ventricular valvular or annular disease of the ventricle being studied, atrial volume loading and pressure differentials, ventricular end-diastolic pressure, ventricular or atrial compliance, or tissue changes associated with scarring or infiltrative diseases of the myocardium.

Most measures of diastolic performance are echocardiography based and incorporate momentary points-in-time measures and relationships often expressed as ratios. While others, such as flow propagation and deceleration time, seek to measure initial flow acceleration and deceleration, respectively. They depend on Doppler techniques that are angle dependent producing velocity related waveforms from a variable orifice inlet into the ventricle. Velocity is an important part of filling but is dependent on orifice size and pressure differential between the volume sources.

With typical echocardiography, the most frequently used measures include the ratio of the pulse-wave Doppler of the LV inflow tract (LVIT) mitral valve (MV) peak early filling velocity (E) to the peak late filling velocity (A) expressed as the E/A ratio; the flow propagation slope (Fp) of the color flow Doppler (CFD M-mode) across the LVIT; the initial closing velocity immediately following the E wave of the LVIT flow expressed as the deceleration time (DT); the pulse tissue Doppler or speckle-tracking (TDI) of the mitral annular peak early filling velocity (E′); and the ratio of the E/E′ and the ratio of the E/Fp.

As of this writing, new techniques may utilize speckle tracking with measures such as longitudinal strain and strain rate. Theses quantify a change in myocardial fibril length or relative position to markers, speckles, within itself providing mechanical changes.

The diastolic filling period can be subdivided into early diastolic filling including isovolemic relaxation time, intermediate diastolic filling, and atrial component including a pre-ejection period. At rest, the majority of diastolic filling normally occurs during the early diastolic filling phase. During exercise the majority of filling shifts to the atrial component. As this occurs an individual will approach or reach their point of exasperation where any increase in heart rate will not produce an increase in minute cardiac output. With increasing degrees of diastolic dysfunction more of the initial filling is shifted to the atrial or active component similarly as if the individual was in some state of activity while actually at rest.

By other volume analysis techniques such as magnetic resonance (MR), computerized tomographic (CT), and nuclear medicine (NM), a volume curve is generated with peak early filling rate (PEFR) and peak early filling volume (PEFV) being expressed in either volume or volume-related terms.

The volume curve represents the sum of all inputs including time, volume, pressure differentials, rates of relaxation and contraction, flow, and compliance. Echocardiography and, more specifically, Doppler measurements are currently the method of choice for evaluation of ventricular diastolic performance. M-mode analysis of the mitral or tricuspid valve waveforms was first used to measure and evaluate tissue motion, excursion, speed, and timing. Measurements included D-E slope, E-F slope, and A-C slope. IRT could also be measured. As is known in the art, the presence of a “B” point was indicative of diastolic dysfunction. Doppler technology is currently the method of choice for evaluating hemodynamics replacing many of the m-mode techniques. Pulse and continuous wave Doppler allow flow velocity evaluation while Tissue Doppler and Speckle-Tracking technologies evaluate rates of volume changes. All are spatially dependent requiring placement in the area of highest velocity and all but Speckle Tracking are angle dependent. All assume homogeneous flow or displacement throughout the orifice or muscle segment. Flow velocities and rates of fill are affected by changes in valve function such as orifice or compliance and segmental muscle disease associated with ischemia and other myopathies. Flow velocity is only one contributor to volume exchange as expressed in the E/A ratio. The rate of pressure equalization between the atria and ventricle as expressed in the DT and flow velocity and rates of tissue displacement combined in measures such as E/E′ are useful but can be misleading with hyper or hypovolemia.

Measures providing a dynamic representation of initial flow-volume/pressure change between the left atrium (LA) and the LV are echoes' Fp and the PEFR of other volume analysis techniques. The nature of these measures is to record the flow or filling velocity deterioration as the blood volume flows into the LV cavity during the initial phase of diastole. Central problems with these techniques are that they are measured across a constantly variable orifice in the diastolic atrial-ventricular valve flow, are measuring a moving flow target with a fixed sampling point or vector reference, or do not consider the relationship with the intermediate or active filling components of LV filling.

Therefore, it would be beneficial to provide a system and method that provide an improved measure in the quantification of the complex relationship of ventricular filling. By way of example, such will be beneficial in clinical assessment of heart failure patients for differentiation of casualty and direction of care qualifying and quantifying diastolic performance, also physiologic testing especially endurance training and cardiac recovery, in women's medicine to better understand hormone protection and HF, scar load post myocardial infarct and the benefit of rehab, the effect of hypertension on ventricular compliance, as well as other restrictive and constrictive cardiomyopathies, and optimizing device therapies such as biventricular pacemakers or other therapies. It can prove useful in pharmaceutical management for diuretics, beta blockers, other medications influencing cardiac function, as well as any diastolic focused medical therapies. A benefit will be realized in studies including cardiac function that provide a capability to specifically describe the diastolic relationship in a numeric form.

### SUMMARY

Embodiments of the present invention are directed to systems and methods for achieving comparison and documentation of diastolic features of both the right ventricle (RV) and the left ventricle (LV). By way of example, initial filing velocity, early filling volume, a ratio of early and intermediate filling velocities, and a computation are used to calculate diastolic filling performance and thus provide significant insight into the LV filling environment. Such data may be used to guide clinical decisions directed towards improving activity capacity in patients with diastolic heart failure (DHF) or diastolic dysfunction (DD) without requiring the patient to be evaluated in a state of physical exertion.

One method aspects of the invention includes evaluating cardiac diastolic function by measuring a volumetric flow of blood through a first heart and determining volume change rates during a diastolic flow period of the volumetric flow. A diastolic index is produced from a combination of the volume change rates. By determining a diastolic index for various hearts and various heart conditions, a comparison of the indices provide a desirable evaluation of the cardiac function of a heart being examined.

Further, by weighting volumetric flow features of the hearts with the associated diastolic index and comparing the weighted values, an enhanced sensitivity is provided for evaluating the cardiac function of the heart.

Volumetric changes and rates of changes are determined using volume-curve analysis of the diastolic phase. An initial filling volume curve vector or tangent is identified, and the slope of an initial filling phase calculated as an initial fill rate (R1). A secondary slope is calculated from an intermediate filling phase vector, best fit line, or tangent displayed after the initial filling phase representative of the intermediate filling rate (R2). Presented as diastolic indices, R1/R2, R2/R1 or R1×R2 volumetric filling relationships may be defined as inputs (orifice size, pressure differentials, relaxation coordination, compliance, and the like). By way of example, R1 may represent the inputs of ventricular relaxation coordination, rates of ventricular relaxation via a Tei Index or Myocardial performance Index (MPI), left atrial preload, atrial-ventricular orifice variables, fluid viscosity and inertial properties, and available ventricular volume. R₂ represents a phase often referred to as diastasis because of the intract-flow waveforms produced by pressure and Doppler waveforms. Flow is occurring during this phase but because of the decreased pressure differential and a less variable atrial-ventricular valve orifice, velocity and pressures change little. It is the phase that is thought of as providing the cardiac reserve but in actuality it is the R1 phase that is “consumed” first as filling responsibilities shift to the intermediate and active components (at least in DD).

One method for evaluating cardiac diastolic function according to the teachings of the present invention may comprise measuring a volumetric change of blood through a heart, determining first and second volume change rates during a diastolic filling period of the volumetric change and formulating a diastolic index from a combination of the first and second volume change rates. Volumetric features may then be weighted by the diastolic index (MI) and evaluated against a preselected value. By way of example, the volumetric change measuring may comprise measuring the volumetric change for a plurality of hearts, each having a predetermined condition selected from hearts having a normal diastolic function, a mild diastolic dysfunction, a moderate diastolic dysfunction, a severe diastolic dysfunction, and may be useful in monitoring specific functional responses such as aerobic training and anaerobic training, thus each having a designated diastolic index, wherein the evaluating comprises comparing the weighted feature for the hearts having the predetermined conditions. Yet further, the volumetric feature may comprise a diastolic filling period (DFP) defined as a difference between a cycle duration and systolic duration, a stroke volume (SV), an HR or cardiac cycle duration (T), an initial filling time (IFT), an ejection fraction (EF) of a ventricle, initial filling volume (IFV), and initial filling volume percent (IFV %), and combinations thereof.

An initial filling time (IFT) is determined as an intersection of the initial filling volume curve vector (R1) and the secondary slope vector (R2) and is a measure of time initiating from the mid-point of the minimal systolic volume and terminating at this described intersection point. A peak initial filling volume or volume percent is determined as the point of the volume curve corresponding to the intersection of the initial filling volume curve vector (R1) and the secondary slope vector (R2) and can be represented either as milliliters or percent of the stroke volume, stroke volume as the difference between the maximum ventricular volume and the minimal ventricular volume expressed in milliliters, diastolic filling period is the measure in milliseconds represented as the cycle duration less the systolic duration. Heart rate is displayed as time and is the HR interval in milliseconds or can be calculated as (1/HR)×60 displayed in milliseconds. The various forms of the index not only provide for disease differentiation, as do many of the other measures previously published, but provide methods of measure or quantification of disease or dysfunction severity which the other known measures can only loosely render. The Moro Index may define the volumetric relationships of diastole as a function of DFP, SV, HR, EF, IFV, IFV % and as a percent of the initial filling time to the diastolic filling period as DV %.

By way of further example, these measures of R1 and R2 may be indexed or weighted against selected volume curve features, such as a diastolic filling period (to provide a performance index herein referred to as a Moro Index_{D}), and further for stroke volume (Moro Index_{SV}), as heart rate or time (Moro Index_{T}), as ejection fraction (Moro Index_{EF}), as initial filling volume (Moro Index_{V}), as a percent of Initial filling volume (Moro Index_{%}), and as a percent of the initial filling time to the diastolic filling period (Moro Index_{DV %}). As a result, a correction for changes in volume loading associated with shifts in diastolic filling dynamics is provided.

The feature parameter may be either multiplied or divided into or by the resulting R₁ and R₂ relationship and may or may not require a constant factor or other function to produce a graphic or numeric measure.

By being able to quantify diastolic dysfunction by various parameters or weighted features, a more precise measure of a specific disease state can be measured. The diastolic filling period (DFP) is defined as a difference between the cycle duration and systolic duration. Using time or heart rate, a relationship including all components of cardiac function is developed. The percent of initial filling volume helps to identify individuals with poor active atrial contributions such as with atrial fibrillation. The stroke volume (SV) feature will correct for changes in volume loading. There may also be iterations that involve the use of multiple factors in various combinations to describe the function.

The present system and method are useful in the diagnosis, stratification of diastolic function and treatment strategies for diastolic heart failure across multiple imaging modalities including CMR, CCT, CNM, and echo or any volume rendering method, providing a global measure of diastolic performance.

One value of this measure is in the quantification of complex relationships of ventricular filling. Systems and methods according to the teachings of the present invention are useful as exemplified such will be beneficial in clinical assessment of heart failure patients for differentiation of causality and direction of care qualifying and quantifying diastolic performance. Examples include physiologic testing especially endurance training and cardiac recovery; women's medicine to better understand hormone protection and HF; scar load post myocardial infarct and the benefit of rehab; the effect of hypertension on ventricular compliance; as well as other restrictive and constrictive cardiomyopathies; and optimizing device therapies such as biventricular pacemakers or carotid body stimulators. Further, benefits will be seen in pharmaceutical management for diuretics and beta blockers as well as any diastolic focused medical therapies. Any study or study of cardiac function will benefit from the ability to specifically describe the diastolic relationship in a numeric form.

### BRIEF DESCRIPTION OF DRAWINGS

Embodiments of the invention are described by way of example with reference to the accompanying drawings in which:

FIGS. 1, 2, 3 and 4 are ventricular volume curves for a heart having normal diastolic function and for hearts having mild, moderate, and severe diastolic dysfunction, respectively;

FIGS. 5 and 6 are ventricular volume curves for hearts having a normal diastolic function, but with an increased heart rate (HR), and a normal diastolic function with increased volume and increased ejection fraction (EF), respectively;

FIG. 5A is a flow chart illustrating one method for evaluating cardiac diastolic function according to the teachings of the present invention, by way of example;

FIGS. 7 and 8 present data in table form and as a plot, respectively, for a diastolic index (R1/R2), herein referred to as an MI of hearts having various diastolic conditions and in various states including resting, increased HR, decreased HR, increased volume and increased EF, increased volume and decreased EF, increased EF, hypovolemia, and hypovolemia and increased HR, by way of illustrative examples;

FIGS. 9 and 10 present data in table form and as a plot, respectively, for a performance index, herein referred to as a weighted Moro Index (MI_{feature}), developed for selected volumetric flow features weighted by dividing the feature by the diastolic index of FIG. 7, and herein presented for the IFV %, and illustrated as MI_{%}, by way of example;

FIGS. 11 and 12 present data in table form and as a plot, respectively, for the Moro Index developed for selected volumetric features weighted by the diastolic index of FIG. 7, and herein presented for the DV % feature weighted by dividing the diastolic index by the feature, and illustrated as MI_{DV %}, by way of example;

FIGS. 13 and 14 present data in table form and as a plot, respectively, for the weighted Moro Index developed for selected volumetric features weighted by the diastolic index (MI) of FIG. 7, and herein presented for the DFP feature weighted by multiplying the diastolic index by the feature, and illustrated as MI_{D}, by way of example;

FIGS. 15 and 16 present data in table form and as a plot, respectively, for a diastolic index (MI) of R2/R1, and herein presented for the IFV % weighted by the MI divided by the IFV % feature, and illustrated as MI_{%} by way of example;

FIG. 17 is a ventricular volume curve for an aerobically trained heart in a resting state illustrating one determination of R1 and R2, by way of example;

FIG. 18 is the ventricular volume curve of FIG. 17 illustrating selected feature values, by way of example;

FIG. 19 is a ventricular volume curve for an anaerobically trained heart in a resting state illustrating one determination of R1 and R2, by way of example;

FIG. 20 is the ventricular volume curve of FIG. 19 illustrating selected feature values, by way of example;

FIGS. 21 and 22, 23 and 24, and 25 and 26 present data in table form and as a plot for MI for various volumetric features observed for a normal heart, aerobic and anaerobic trained hearts, and hearts having mild, moderate and severe diastolic dysfunction, wherein the diastolic index (MI) is R1/R2. by way of example; and

FIG. 27 is a diagrammatical illustration of a system for evaluating cardiac diastolic function according to the teachings of the present invention, herein presented by way of example.

### DETAILED DESCRIPTION OF EMBODIMENTS

A description of embodiments of the invention will now be described more fully hereinafter with reference to the accompanying drawings, in which the embodiments are shown by way of illustration and example. This invention may, however, be embodied in many forms and should not be construed as limited to the embodiments set forth herein. Rather, these embodiments are provided so that this disclosure will be thorough and complete, and will fully convey the scope of the invention to those skilled in the art. Like numerals refer to like elements.

With reference initially to FIGS. 1-4, differences in ventricular volume curves in a range of normal diastolic function to severe dysfunction are herein illustrated by way of example. An examination of these volume curves reveals that, during a period encompassing initial filling to intermediate filling, the slopes represented by initial filling rate (R1) and intermediate filling rate (R2) change dramatically, as do the initial filling volume (IFV). The initial filling time (IFT) changes, but not as dramatically.

The diastolic index includes the relationship of the initial filling slope of “A” (R1) to the intermediate filling slope of “B” (R2). The change in volume and change in time (dV/dt) of the “best fit” line for the “A” segment of the curve is known as “R1”, the dV/dt of the “best fit” line for the “B” segment of the curve is known as “R2”. By relating R1 to R2 a single number is generated that describes the passive ventricular filling phase. Iterations would include R1/R2 as presented within the graphic displays but could also include R2/R1 or R1×R2 as included in the tables for comparison. The diastolic index may be used as the numerator or denominator of a weighted measure.

The measurements and calculations displayed within the graphic displays are the R1 and R2 (components of the diastolic index) and the weighted measurements, herein described as the weighted Moro Index of volumetric features (MI_{feature}) including stroke volume (SV), Initial Filling Volume (IFV), and the Initial Filling Time (IFT), by way of example. The SV is the difference between the maximum and minimum ventricular volumes. The IFV is the volume change in the ventricle from the beginning of filling following systole to the point on the volume curve that corresponds in time to the intersection of R1 and R2 expressed in liters (1 L=1.000), and the initial filling time (IFT) is the time from the mid-point of the peak minimal systolic volume of S to the determination of initial filling volume (IFV) expressed in seconds (1 s=1.000).

Measurements that are not displayed but are used in weighted calculations and recorded are ventricular cycle time (T), systolic time (S), and diastolic filling period (DFP). HR is measured from the beginning of ventricular ejection to the end of the isovolemic contraction (ICT) phase represented as the end of the curve and is expressed in seconds to the thousandth (1 s=1.000). S is defined as the time from the start of ejection to the mid-point of the trough created at end systolic volume and is expressed in seconds to the thousandth (1 s=1.000). DFP is defined as the remainder of the ventricular waveform following the S interval and extends to the end of the ICT.

Volumetric calculations displayed are ventricular Ejection Fraction (EF %) and Initial Filling Volume Percent (IFV %). The EF % is the SV divided by the maximum volume multiplied by 100. The IFV % is the IFV divided by the SV and multiplied by 100.

Calculations not displayed are associated with examples of various weighting types and methods. Moro Index DV % (MI_{DV %}) requires calculating the ratio of IFT to DFP to define the index. By way of example, D (used for DFP), T (or HR), SV, EF, V (used for IFV), and IFV % or any other features or factors are determined for weighted index calculations, wherein T=(1/HR)×60. By way of example, these relationships may be represented as follows:

MI_{T}=MI/(60/HR)

MI_{D}=MI/DFP

MI_{DV %}=MI/DV %

MI_{SV}=MI/SV

MI_{V}=MI/IFV

MI_{%}=MI/IFV %

MI_{EF}=MI/EF %

The examples presented in the graphic displays may include, but are not limited to, various multipliers, coefficients, and/or addition of constants or other mathematical functions or processes without diminishing the core of the relationship being the diastolic index. The graphic displays include several other iterations.

The comparison tables included in the illustrations include examples of hemodynamic states frequently encountered to demonstrate the differentiation between the numeric descriptions of the volume curves and the associated calculations. The comparison tables and plots provide a snapshot of examples allowing comparison between different levels of severity and within a diastolic function class. The hemodynamic states include Resting, Increased Heart Rate, Decreased Heart Rate, Increased Volume, Decreased Volume, Increased Ejection Fraction, and various combinations to demonstrate a compounding effect. The classes of diastolic performance include Normal, Mild Diastolic Dysfunction, Moderate Diastolic Dysfunction, Severe diastolic Dysfunction, and a comparison between Normal, Aerobically, and Anaerobically trained Athletes.

By way of example, the volume curves displayed in FIGS. 1-4 are intended to highlight the diastolic component of the cardiac cycle. However, diastolic dysfunction and diastolic heart failure can exist with normal systolic function as presented or with abnormal systolic function and systolic dysfunction and systolic heart failure can exist with normal diastolic function. The cardiac cycle is a continuum with the previous diastolic event affecting the current systolic in turn affecting the current diastolic and the following systolic component. The effect of systolic performance on the diastolic curve does not change the diagnostic or clinical value of the resulting relationships.

The volume curves are formatted with the y-axis (vertical) representing volume in milliliters or normalized as may be appropriate. The scale begins at the end-systolic volume with the peak of the scale representing the end-diastolic volume. The x-axis (horizontal) is time in milliseconds beginning at zero and extending to the end of the curve. By way of illustration, the volume curves start at the beginning of the ventricular ejection phase and terminate just prior to the beginning of the next cardiac cycle's ejection phase. The isovolemic and pre-ejection phases are included but not illustrated or defined in these drawings. The isovolemic relaxation (IRT) phase is known to be affected by atrial preload and ventricular relaxation coordination but any changes in these parameters would have a corresponding effect on diastolic function directly impacting the R1 component of the diastolic index.

As will be appreciated by those of skill in the art, the details presented in the curves will be dependent on the number of sample points taken through each phase of the cardiac cycle. The greater the number of samplings during any particular phase, the higher the fidelity of the curve during that phase. The effect of higher sampling rates would be to increase specificity and variability within the curve. Simple physiologic functions and activities can affect volume returns to the ventricles such as breathing and talking creating dynamic changes to pressures and volumes within the heart that can be reflected in the resulting volume curve either requiring smoothing techniques or the use of “best fit” lines, or control of physiologic input.

With continued reference to FIGS. 1-4, the curves are divided into 4 phases for descriptive purposes identified as S, A, B, and P. The S phase is the systolic ejection phase of the ventricle which herein begins at time zero with the initial change in ventricular volume and terminates at the middle of the “trough” created which may include some portion of the IRT. The A phase is the early diastolic filling of the ventricle and begins at the termination point of the S and terminates during the transition to the next phase determined by the intersection of slope line generated as a “best fit” for the A phase and the following phase B. The B phase, often referred to as the “period of diastasis”, begins from the point of the A phase termination and extends to the beginning of the active atrial contribution if present. The P phase represents the active atrial contraction and includes the pre-ejection period and isovolemic contraction period. The P phase extends from the end of the B phase terminating with the start of the next S component. The P phase may be omitted in atrial and atrial sinus disease states such as atrial fibrillation. The normal ventricular volume curve is presented with a heart rate of 74 BPM. Approximately 80% of the filling volume is delivered during the initial rapid filling A phase, with the remaining volume contributed during the atrial contraction. The B phase contributes very little volume and is often referred to as the period of diastasis. By way of example, the representation of the volume curve of FIG. 1 is intended as a basis of comparison to various normal and abnormal volume curves herein illustrated.

For illustration purposes, the curves herein presented are formatted to minimize variability not specific or directly associated to diastolic performance for the study state in order to better illustrate the resulting evaluation using the Moro Index unique to the diastolic filling pattern and the results of weighting the Moro Index. Ventricular end diastolic volume, SV, EF %, and heart rate (HR) are held constant within a study state whenever possible to better illustrate the effects of manipulating select variables for ease of comparison. To illustrate these effects, ventricular volume curves have been reviewed for Resting Volume, Increased Heart rate, Decreased Heart rate, Increased Volume and Increased Ejection Fraction, Increased Volume and Decreased Ejection Fraction, Increased Ejection Fraction, Hypovolemia, and Hypovolemia and Increased Heart rate as sample combinations to demonstrate compounding effects with examples of Normal Diastolic Function, Mild Diastolic Dysfunction, Moderate Diastolic Dysfunction, and Severe Diastolic Dysfunction. Examples are herein presented.

With reference now to FIGS. 5 and 5A, consider one method 100 for evaluating cardiac diastolic function to include measuring the first volumetric changes of blood volume through a first heart 102, wherein the first heart includes a first predetermined condition, such as a heart having a normal diastolic function, but with an increased heart rate (HR). Initial and intermediate volume change rates (R1 and R2, respectively) during the diastolic filling period (A) of the first volumetric are determined 104. As earlier illustrated with reference to FIG. 1, the intermediate volume change rate (R2) follows the initial volume change rate (R1). A first diastolic index (MI) is formed 106 from a combination of the initial and intermediate volume change rates of the first volumetric flow (R1/R2, R2/R1, or R1×R2 by way of example) and a first value determined. Volumetric features are determined 108 and weighted by the diastolic index 110 selected

Further, with continued reference to FIG. 5A and with reference to FIG. 6, by repeating the measurement process 110, a second volumetric change of blood through a second heart is measured, wherein the second heart includes a second predetermined condition, such as a heart having a normal diastolic function, but with an increased volume and an increased ejection fraction (EF). Initial and intermediate volume change rates (R1 and R2, respectively) during the diastolic filling period (A) of the second volumetric changes of blood are determined. As illustrated, the intermediate volume change rate (R2) follows the initial volume change rate (R1). A second value for the selected diastolic index is formed.

The diastolic index (MI) may be formed by dividing the initial volume change rate (R1) by the intermediate volume change rate (R2), dividing the intermediate volume change rate by the initial volume change rate, and multiplying the initial volume change rate by the intermediate volume change rate. Further, the predetermined conditions of the hearts may include hearts having normal diastolic function, mild diastolic dysfunction, moderate diastolic dysfunction, and severe diastolic dysfunction, as herein presented by way of example.

As further illustrated with continued reference to FIG. 5A, the above process may be completed for hearts having known predetermined conditions and diagnostic indices determined, such as illustrated with reference to the Table of FIG. 7 for normal, mild, moderate and severe conditions during Resting Volume, Increased Heart rate, Decreased Heart rate, Increased Volume and Increased Ejection Fraction, Increased Volume and Decreased Ejection Fraction, Increased Ejection Fraction, Hypovolemia, and Hypovolemia and Increased Heart rate, and compared 114, by way of non-limiting examples. As illustrated in FIG. 7, the diastolic index selected for comparison is R1/R2, by way of example. FIG. 8 is a plot of the values in table of FIG. 7 for further illustration and comparison.

In one method according to the teachings of the present invention, the step 104 of determining the R1 and R2 for a first heart is repeated for a second heart or multiple hearts and the indices compared for the various hearts and various heart conditions. By way of example, such a method includes measuring a volumetric flow of blood through a first heart and determining volume change rates during a diastolic flow period of the volumetric flow to establish a diastolic index for the heart under examination. Volume change rates during the diastolic flow period in second or multiple hearts are determined and second or multiple diastolic indices established. The diastolic index for the heart under examination is then compared to the diastolic index of the second heart or diastolic indices of the multiple hearts having various known heart conditions for evaluating the cardiac function of the heart under examination.

By way of example and with continued reference to FIGS. 7 and 8, it will be appreciated that for the hearts under consideration, there is a clearly measurable sensitivity in resting, decreased heart rate and increased heart rate with increased ejection fraction states for a heart having a normal diastolic function to that having a mild diastolic dysfunction, and thus desirable for monitoring and comparing the diastolic index (MI) for the heart under examination.

The process may then be continued, as above described with reference to FIG. 5A by weighting a volumetric flow feature or features of the first heart with its diastolic index to provide a measure of diastolic filling performance and comparing the weighted diastolic index of the first heart with the second or multiple weighted diastolic indices for evaluating the cardiac function of the first heart to the diastolic indices of hearts having known conditions.

By way of further example, and with continued reference to FIG. 8, one of skill in the art would appreciate that a comparison between a normal heart and a heart having a mild diastolic dysfunction is better compared for those hearts having a decreased HR, increased volume and increased EF, and increased volume and decreased EF. wherein differences therebetween are more pronounced, and thus sensitivity using R1/R2 enhanced.

For further analysis and comparison, methods according to the teachings of the present invention include preselecting features of the volumetric curve of interest, such as a diastolic filling period (D) defined as the difference between the cycle duration and systolic duration, a stroke volume (SV), an HR or cardiac duration (T), an initial filling volume (V), an ejection fraction (EF) of a ventricle, an initial filling volume percent (%), and a percentage (DV %) of the initial filling time of a total diastolic filling period and quantifying values for the heart conditions being examined, such as those identified with reference again to FIG. 7. The preselected features are then weighted with the selected diastolic index to form, what is herein referred to as a Moro Index weighted with a feature of the volumetric curve (MI_{feature}).

As desired, there may be an MI_{feature} for each feature, thus MI_{D}, MI_{V}, MI_{T}, MI_{EF}, MI_{SV}, MI_{%}, and MI_{DV %} or as desired as above described. The MI_{feature} may comprise dividing the diastolic index MI by the preselected feature, dividing the preselected feature by the MI, or multiplying the MI by the preselected feature to arrive at the weighted values, by way of example for the diastolic index R1/R2 addressed with reference to FIGS. 7 and 8, and examples of features divided by R1/R2 for MI % and MI_{DV %} illustrated with reference to FIGS. 9 and 10 and FIGS. 11 and 12, respectively. As above addressed, and with continued reference to FIGS. 10 and 11 by way of example, weighting as herein presented would be useful for evaluating the heart having moderate to severe diastolic dysfunction with hypovolemia and increased HR, but not for increased HR alone, as emphasized by the behavior illustrated in FIG. 12 comparing moderate to mild and severe conditions.

The indices herein presented by way of example demonstrated an exponential or logarithmic relationship with greatest variability noted in the normal range and the least in the severe range when using the R1/R2 calculation.

As will be appreciated by those of skill in the art, now having the benefit of the teachings of the present invention, comparing various weighting combinations will prove useful for various heart evaluations. By way of further example, FIGS. 13 and 14 illustrates weighting of DFP×R1/R2 resulting is a desirable sensitivity comparison between normal and mild conditions for a resting heart, one having increased volume and decreased EF, and decreased HR. FIGS. 15 and 16 illustrate a weighting of R2/R1 divided by the feature, IFV % resulting in a desirable sensitivity comparison between moderate and severe conditions for a resting heart, but not for comparing the moderate heart with mild or severe with an increased HR or hypovolemia and increased HR.

Volume management is a component of pharmacologic clinical control of hydration that can benefit from volume curve analysis using the Moro Index and the associated weighted measures. Increases in HR will have a dramatic effect on diastolic filling and the subsequent SV with hypovolemia and/or diastolic dysfunction diminishing early diastolic filling phase contribution.

By way of further example, ventricular end diastolic volume, stroke volume (SV), percentage ejection fraction as a percentage of maximal ventricular volume (EF %), and heart rate (HR) are held constant for an Anaerobic Trained Athlete, and Aerobic Trained Athlete to demonstrate the effect of diastolic performance and cardiac function between each of the training methods.

The volume curves represent the sum of all inputs including time, volume, pressure differentials, flow and compliance. Because diastolic performance is dependent on preload and preload is dependent on volume, volume analysis must accompany any diastolic analysis.

Consider the following example for a Normal Diastolic Function with Increased Heart Rate, as illustrated with reference again to FIG. 5, and an effect of increased heart rate on diastolic filling period (DFP). The increase in heart rate decreases the filling time of the atria lowering the preload and reducing the initial pressure differential between the atria and the ventricle. The initial filling volume percent of the total filling begins to decrease and some of the filling requirements are shifted to the B phase. Such a representation is useful for comparison with Normal Diastolic Function, Normal Diastolic Function with Decreased Heart Rate, Mild Diastolic Dysfunction with Increased Heart Rate, Moderate Diastolic Dysfunction with Increased Heart Rate, and Severe Diastolic Dysfunction with Increased Heart Rate, by way of example.

For a Normal Diastolic Function with Decreased Heart Rate and the effect of decreased heart rate on the filling curve, a similar curve to the Normal results, but with a longer timeline. The initial filling volume and percent are normal with an increase noted in the time component of the “B” phase as the ventricle has a longer time to relax and more volume is made available from the atria. This representation is useful for comparison with Normal Diastolic Function, Normal Diastolic Function with Increased Heart Rate, Mild Diastolic Dysfunction with Decreased Heart Rate, Moderate Diastolic Dysfunction with Decreased Heart Rate, and Severe Diastolic Dysfunction with Decreased Heart Rate.

For a Normal Diastolic Function with Increased Volume presented in high input/output states such as valvular insufficiency, septal defects or artero-venous shunts, ventricular relaxation continues into the A phase and may be limited by the A-V valve orifice or atrial volume. Filling of the ventricle continues throughout diastole with the B phase providing a greater contribution than normal. This representation is useful for comparison with Normal Diastolic Function, Normal Diastolic Function with Increased Volume and Increased Ejection Fraction, Normal Diastolic Function with Increased Volume and Decreased Ejection Fraction, Mild Diastolic Dysfunction with Increased Volume and Increased Ejection Fraction, Mild Diastolic Dysfunction with Increased Volume and Decreased Ejection Fraction, Moderate Diastolic Dysfunction with Increased Volume and Increased Ejection Fraction, Moderate Diastolic Dysfunction with Increased Volume and Decreased Ejection Fraction, Severe Diastolic Dysfunction with Increased Volume and Increased Ejection Fraction, and Severe Diastolic Dysfunction with Increased Volume and Decreased Ejection Fraction, by way of example

For a Normal Diastolic Function with Increased Ejection Fraction, even a relatively minor change to the dV/dt of R1 will dramatically change the Moro Index. While curves may appear to be benign, they can demonstrate a deficiency in early volume delivery. This representation is for comparison with Normal Diastolic Function, Normal Diastolic Function with Increased Volume and Increased Ejection Fraction, Normal Diastolic Function with Increased Volume and Decreased Ejection Fraction, Mild Diastolic Dysfunction with Increased Ejection Fraction, Moderate Diastolic Dysfunction with Increased Ejection Fraction, and Severe Diastolic Dysfunction with Increased Ejection Fraction.

For a Normal Diastolic Function with Increased Volume and Increased Ejection Fraction, as illustrated with reference again to FIG. 6, the effect SV and EF may have on the volume curve and thus performance is dependent on the ability of the ventricle to fill. Increased SV or volume change requires an increase in filling flow-delivery and relaxation-compliance characteristics. This representation is for comparison with Normal Diastolic Function, Normal Diastolic Function with Increased Volume and Decreased Ejection Fraction, Mild Diastolic Dysfunction with Increased Volume and Increased Ejection Fraction, Moderate Diastolic Dysfunction with Increased Volume and Increased Ejection Fraction, and Severe Diastolic Dysfunction with Increased Volume and Increased Ejection Fraction.

For Normal Diastolic Function with Increased Volume and Decreased Ejection Fraction, the effect of decreased EF associated with systolic heart failure with normal diastolic function may be represented by a normal Moro Index, wherein Diastolic performance is not independent of systolic but poor systolic performance is not predictive of diastolic function. This representation is for comparison with Normal Diastolic Function, Normal Diastolic Function with Increased Volume and Increased Ejection Fraction, Mild Diastolic Dysfunction with Increased Volume and Decreased Ejection Fraction, Moderate Diastolic Dysfunction with Increased Volume and Decreased Ejection Fraction, and Severe Diastolic Dysfunction with Increased Volume and Decreased Ejection Fraction.

For Normal Diastolic Function with Hypovolemia, an effect may be observed with volume management and dehydration on cardiac filling. The IFV % decreases. There is less atrial preload resulting in lower pressure differentials during the “A” phase. The “B” phase contributes a greater than normal volume to the ventricular filling affecting cardiac functional reserve. This representation is useful for comparison with Normal Diastolic Function, Normal Diastolic Function with Hypovolemia and Increased Heart Rate, Mild Diastolic Dysfunction with Hypovolemia, Mild Diastolic Dysfunction with Hypovolemia and Normal Compliance, Moderate Diastolic Dysfunction with Hypovolemia, Moderate Diastolic Dysfunction with Hypovolemia and Normal Compliance, Severe Diastolic Dysfunction with Hypovolemia, and Severe Diastolic Dysfunction with Hypovolemia and Normal Compliance.

For Normal with Hypovolemia and Increased Heart Rate, the effect of lower blood volume may be present with reduced returning volume to the atria or A-V flow restriction with the addition of increasing the heart rate, decreasing the DFP and further reducing atrial filling and preload. The increased heart rate has a more pronounced effect on ventricular filling duration and volume than on ejection time. This representation is for comparison with Normal Diastolic Function, Normal Diastolic Function with Hypovolemia, Mild Diastolic Dysfunction with Hypovolemia, Moderate Diastolic Dysfunction with Hypovolemia, Moderate Diastolic Dysfunction with Hypovolemia and Normal Compliance, and “Severe Diastolic Dysfunction with Hypovolemia.

For Mild Diastolic Dysfunction, the effect of even mild diastolic dysfunction can be seen using the Moro Index. The effect can be similar to what is observed with hypovolemia and normal diastolic function. The curve may be compared with the Normal Diastolic Function, Moderate Diastolic Dysfunction, Severe Diastolic Dysfunction and the volume curves within the Mild Diastolic Dysfunction set.

For Mild Diastolic Dysfunction with Increased Heart Rate, the effect of increased heart rate on DFP with mild diastolic dysfunction is observed, wherein the IFV % of the DFP decreases more than observed with normal diastolic function at a similar heart rate and more of the filling requirements are shifted to the “B” phase. By way of example, this representation is useful for comparison with Normal Diastolic Function, Mild Diastolic Dysfunction, Mild Diastolic Dysfunction with Decreased Heart Rate, Moderate Diastolic Dysfunction with Increased Heart Rate, and Severe Diastolic Dysfunction with Increased Heart Rate.

For Mild Diastolic Dysfunction with Decreased Heart Rate, the effect of decreased heart rate on the filling curve may be somewhat similar to the Mild Diastolic Dysfunction, but with a longer timeline. The IFV and IFV % are similar to Mild Diastolic Dysfunction with an increase noted in the time component of the “B” phase as the ventricle has a longer time to relax and more volume is made available. The result could be a normal SV and EF masking an exertion or rate related malady which becomes evident with the MI evaluation. This representation is useful for comparison with Normal Diastolic Function, Normal Diastolic Function with Decreased Heart Rate, Mild Diastolic Dysfunction, Mild Diastolic Dysfunction with Increased Heart Rate, Moderate Diastolic Dysfunction with Decreased Heart Rate, and Severe Diastolic Dysfunction with Decreased Heart Rate.

For Mild Diastolic Dysfunction with Increased Ejection Fraction, increased EF can have an effect on diastolic dysfunction. These features are often present with hypertensive or hypertrophic myopathies and hypovolemia. This representation is for comparison with Normal Diastolic Function, Normal Diastolic Function with Increased Ejection Fraction, Mild Diastolic Dysfunction, Mild Diastolic Dysfunction with Increased Volume and Increased Ejection Fraction, Moderate Diastolic Dysfunction with Increased Ejection Fraction, and Severe Diastolic Dysfunction with Increased Ejection Fraction.

For Mild Diastolic Dysfunction with Increased Volume and Increased Ejection Fraction, SV and EF may affect the volume curve. Diastolic performance is dependent on the ability of the ventricle to fill. Increased SV or volume change requires an increase in filling flow-delivery and/or relaxation-compliance characteristics. This representation is useful for comparison with Normal Diastolic Function, Normal Diastolic Function with Increased Volume and Increased Ejection Fraction, Mild Diastolic Dysfunction, Mild Diastolic Dysfunction with Increased Volume and Decreased Ejection Fraction, Moderate Diastolic Dysfunction with Increased Volume and Increased Ejection Fraction, and Severe Diastolic Dysfunction with Increased Volume and Increased Ejection Fraction.

For Mild Diastolic Dysfunction with Increased Volume and Decreased Ejection Fraction, an effect may be realized for decreased EF associated with systolic heart failure with mild diastolic dysfunction. Diastolic performance is not independent of systolic but poor systolic performance is not predictive of diastolic function. This representation is for comparison with Normal Diastolic Function, Normal Diastolic Function with Increased Volume and Decreased Ejection Fraction, Mild Diastolic Dysfunction, Mild Diastolic Dysfunction with Increased Volume and Increased Ejection Fraction, Moderate Diastolic Dysfunction with Increased Volume and Decreased Ejection Fraction, and Severe Diastolic Dysfunction with Increased Volume and Decreased Ejection Fraction.

For Mild Diastolic Dysfunction with Hypovolemia, an effect may be observed with volume management and dehydration on cardiac filling. The IFV % decreases compared to normally hydrated examples such as Mild Diastolic Dysfunction. There is less atrial preload resulting in lower pressure differentials during the “A” phase. The “B” phase contributes a greater than normal volume to the ventricular filling affecting cardiac functional reserve. This representation is for comparison with Normal Diastolic Function, Normal Diastolic Function with Hypovolemia, Mild Diastolic Dysfunction, Mild Diastolic Dysfunction with Hypovolemia and Normal Compliance, Moderate Diastolic Dysfunction with Hypovolemia, and Severe Diastolic Dysfunction with Hypovolemia.

For Mild Diastolic Dysfunction with Hypovolemia and Normal Compliance, an effect may be observed with volume management and dehydration on cardiac filling with normal ventricular compliance and relaxation dynamics depicting mild diastolic dysfunction. There may be complex relationships related to myocardial stretch, active relaxation, compliance and other factors combined with atrial preload that will affect the “A” phase. A leftward shift of the IFT is realized for comparison to the moderate and severe illustrations included, but the result could also be a neutral or rightward shift with an accompanying decrease in IFV and IFV %. The transition to the “B” phase may not be clearly defined as the volumetric change may occur more slowly. The “B” phase contributes a greater than normal volume to the ventricular filling affecting cardiac functional reserve. This representation is useful for comparison with Normal Diastolic Function, Normal Diastolic Function with Hypovolemia, Mild Diastolic Dysfunction, Mild Diastolic Dysfunction with Hypovolemia, Moderate Diastolic Dysfunction with Hypovolemia and Normal Compliance, and Severe Diastolic Dysfunction with Hypovolemia and Normal Compliance.

For Moderate Diastolic Dysfunction, a moderate effect on the Moro Index is realized for diastolic dysfunction. The effect can be similar to what is observed with hypovolemia and mild diastolic dysfunction. This curve is meant to be compared with the Normal Diastolic Function, Mild Diastolic Dysfunction, Severe Diastolic Dysfunction and the volume curves within the Moderate Diastolic Dysfunction set.

For Moderate Diastolic Dysfunction with Increased Heart Rate, the effect of increased heart rate on filling time with moderate diastolic dysfunction can be observed. The IFV % of the total filling decreases further compared to mild diastolic dysfunction at the same heart rate and more of the filling requirements are shifted to the “B” phase. This representation is useful for comparison with Normal Diastolic Function, Mild Diastolic Dysfunction with Increased Heart Rate, Moderate Diastolic Dysfunction, Moderate Diastolic Dysfunction with Decreased Heart Rate, and Severe Diastolic Dysfunction with Increased Heart Rate.

For Moderate Diastolic Dysfunction with Decreased Heart Rate, an effect of decreased heart rate on the filling curve may be observed, wherein a similar curve appears for the Moderate Diastolic Dysfunction, but with a longer timeline. The IFV and IFV % are similar to Moderate Diastolic Dysfunction with an increase noted in the time component of the “B” phase as the ventricle has a longer time to relax and more volume is made available from the atria. The result could be a normal SV and EF masking an exertional or rate related malady which becomes evident with the MI evaluation. This representation is for comparison with Normal Diastolic Function, Normal Diastolic Function with Decreased Heart Rate, Mild Diastolic Dysfunction with Decreased Heart Rate, Moderate Diastolic Dysfunction, Moderate Diastolic Dysfunction with Increased Heart Rate, and Severe Diastolic Dysfunction with Decreased Heart Rate.

For Moderate Diastolic Dysfunction with Increased Ejection Fraction, an increased EF can have an effect on diastolic function. These features are often present with hypertensive or hypertrophic myopathies and hypovolemia. This representation is for comparison with Normal Diastolic Function, Normal Diastolic Function with Increased Ejection Fraction, Mild Diastolic Dysfunction with Increased Ejection Fraction, Moderate Diastolic Dysfunction, Moderate Diastolic Dysfunction with Increased Volume and Increased Ejection Fraction, and Severe Diastolic Dysfunction with Increased Ejection Fraction.

For Moderate Diastolic Dysfunction with Increased Volume and Increased Ejection Fraction, the SV and EF may have an effect on the volume curve. Diastolic performance is dependent on the ability of the ventricle to fill. Increased SV or volume change requires an increase in filling flow-delivery and/or relaxation-compliance characteristics. This representation is for comparison with Normal Diastolic Function, Normal Diastolic Function with Increased Volume and Increased Ejection Fraction, Mild Diastolic Dysfunction with Increased Volume and Increased Ejection Fraction, Moderate Diastolic Dysfunction, Moderate Diastolic Dysfunction with Hypovolemia, Moderate Diastolic Dysfunction with Hypovolemia and Normal Compliance, Moderate Diastolic Dysfunction with Increased Ejection Fraction, and Severe Diastolic Dysfunction with Increased Volume and Increased Ejection Fraction.

For Moderate Diastolic Dysfunction with Increased Volume and Decreased Ejection Fraction, an effect of decreased EF associated with systolic heart failure with moderate diastolic dysfunction may be observed. Diastolic performance is not independent of systolic but poor systolic performance is not predictive of diastolic function. This representation is for comparison with Normal Diastolic Function, Normal Diastolic Function with Increased Volume and Decreased Ejection Fraction, Mild Diastolic Dysfunction with Increased Volume and Decreased Ejection Fraction, Moderate Diastolic Dysfunction, Moderate Diastolic Dysfunction with Hypovolemia, Moderate Diastolic Dysfunction with Hypovolemia and Normal Compliance, Moderate Diastolic Dysfunction with Increased Volume and Increased Ejection Fraction, and Severe Diastolic Dysfunction with Increased Volume and Decreased Ejection Fraction.

For Moderate Diastolic Dysfunction with Hypovolemia, an effect may be observed with volume management and dehydration on cardiac filling. The IFV % decreases compared to normally hydrated examples such as “Moderate Diastolic Dysfunction”. There is less atrial preload resulting in lower pressure differentials during the “A” phase. The “B” phase contributes a greater than normal volume to the ventricular filling affecting cardiac functional reserve. This representation is for comparison with Normal Diastolic Function, Normal Diastolic Function with Hypovolemia, Mild Diastolic Dysfunction with Hypovolemia, Moderate Diastolic Dysfunction, Moderate Diastolic Dysfunction with Hypovolemia and Normal Compliance, and Severe Diastolic Dysfunction with Hypovolemia.

For Moderate Diastolic Dysfunction with Hypovolemia and Normal Compliance, an effect that may be observed with volume management and dehydration on cardiac filling with normal ventricular compliance and relaxation dynamics depicting moderate diastolic dysfunction. There may be complex relationships related to myocardial stretch, active relaxation, compliance and other factors combined with atrial preload that will affect the “A” phase. This illustration demonstrates a leftward shift of the IFT, for comparison to the milder and more severe illustrations included, but the result could also be a neutral or rightward shift with an accompanying decrease in IFV and IFV %. The transition to the “B” phase may not be clearly defined as the volumetric flow rate may change more slowly. The “B” phase contributes a greater than normal volume to the ventricular filling affecting cardiac functional reserve. This representation is useful for comparison with Normal Diastolic Function, Normal Diastolic Function with Hypovolemia, Mild Diastolic Dysfunction with Hypovolemia and Normal Compliance, Moderate Diastolic Dysfunction, Moderate Diastolic Dysfunction with Hypovolemia, and Severe Diastolic Dysfunction with Hypovolemia and Normal Compliance.

Observed results for a Severe Diastolic Dysfunction can be similar to what is observed with hypovolemia and moderate diastolic dysfunction. Comparisons are useful with Normal Diastolic Function, Mild Diastolic Dysfunction, Moderate Diastolic Dysfunction and the volume curves within the Severe Diastolic Dysfunction set.

For Severe Diastolic Dysfunction with Increased Heart Rate, an effect of increased heart rate on filling time with severe diastolic dysfunction may be useful. The IFV % of the total filling decreases further compared to moderate diastolic dysfunction at the same heart rate and more of the filling requirements are shifted to the “B” phase. This representation is for comparison with Normal Diastolic Function, Mild Diastolic Dysfunction with Increased Heart Rate, Moderate Diastolic Dysfunction with Increased Heart Rate, Severe Diastolic Dysfunction, and Severe Diastolic Dysfunction with Decreased Heart Rate.

For Severe Diastolic Dysfunction with Decreased Heart Rate, an effect of decreased heart rate on the filling curve is observed, wherein a similar curve is realized for the Severe Diastolic Dysfunction, but with a longer timeline. The IFV and IFV % are similar to “Severe Diastolic Dysfunction” with an increase noted in the time component of the “B” phase as the ventricle has a longer time to relax and more volume is made available from the atria. The result could be a normal SV and EF masking an exertional or rate related malady which becomes evident with the MI evaluation. This representation is for comparison with Normal Diastolic Function, Normal Diastolic Function with Decreased Heart Rate, Mild Diastolic Dysfunction with Decreased Heart Rate, Moderate Diastolic Dysfunction with Decreased Heart Rate, Severe Diastolic Dysfunction, and Severe Diastolic Dysfunction with Increased Heart Rate.

For Severe Diastolic Dysfunction with Increased Ejection Fraction, there is an effect of increased EF on severe diastolic dysfunction. These features are often present with hypertensive or hypertrophic myopathies and hypovolemia. This representation is for comparison with Normal Diastolic Function, Normal Diastolic Function with Increased Ejection Fraction, Mild Diastolic Dysfunction with Increased Ejection Fraction, Moderate Diastolic Dysfunction with Increased Ejection Fraction, Severe Diastolic Dysfunction, and Severe Diastolic Dysfunction with Increased Volume and Increased Ejection Fraction.

For Severe Diastolic Dysfunction with Increased Volume and Increased Ejection Fraction, there may be an effect of SV and EF observed for the volume curve. Diastolic performance is dependent on the ability of the ventricle to fill. Increased stroke volume or volume change requires an increase in filling flow-delivery and/or relaxation-compliance characteristics. This representation is for comparison with Normal Diastolic Function, Normal Diastolic Function with Increased Volume and Increased Ejection Fraction, Mild Diastolic Dysfunction with Increased Volume and Increased Ejection Fraction, Moderate Diastolic Dysfunction with Increased Volume and Increased Ejection Fraction, Severe Diastolic Dysfunction, Severe Diastolic Dysfunction with Hypovolemia, Severe Diastolic Dysfunction with Hypovolemia and Normal Compliance, and Severe Diastolic Dysfunction with Increased Ejection Fraction.

For Severe Diastolic Dysfunction with Increased Volume and Decreased Ejection Fraction an effect is observed for decreased EF associated with systolic heart failure and severe diastolic dysfunction. Diastolic performance is not independent of systolic but poor systolic performance is not predictive of diastolic function. This representation is for comparison with Normal Diastolic Function, Normal Diastolic Function with Increased Volume and Decreased Ejection Fraction, Mild Diastolic Dysfunction with Increased Volume and Decreased Ejection Fraction, Moderate Diastolic Dysfunction with Increased Volume and Decreased Ejection Fraction, Severe Diastolic Dysfunction, Severe Diastolic Dysfunction with Hypovolemia, Severe Diastolic Dysfunction with Hypovolemia and Normal Compliance, and Severe Diastolic Dysfunction with Increased Volume and Increased Ejection Fraction.

For Severe Diastolic Dysfunction with Hypovolemia, an effect may be observed with volume management and dehydration on cardiac filling. The IFV % decreases compared to normally hydrated examples such as Severe Diastolic Dysfunction. There is less atrial preload resulting in lower pressure differentials during the “A” phase. The “B” phase contributes a greater than normal volume to the ventricular filling affecting cardiac functional reserve. This representation is for comparison with Normal Diastolic Function, Normal Diastolic Function with Hypovolemia, Mild Diastolic Dysfunction with Hypovolemia, Moderate Diastolic Dysfunction with Hypovolemia, Severe Diastolic Dysfunction, and Severe Diastolic Dysfunction with Hypovolemia and Normal Compliance.

For Severe Diastolic Dysfunction with Hypovolemia and Normal Compliance, an effect may be observed with volume management and dehydration on cardiac filling with normal ventricular compliance and relaxation dynamics depicting severe diastolic dysfunction. There may be complex relationships related to myocardial stretch, active relaxation, compliance and other factors combined with atrial preload that will affect the “A” phase. This illustration demonstrates a leftward shift of the IFT, for comparison to the mild and moderate illustrations included, but the result could also be a neutral or rightward shift with an accompanying decrease in IFV and IFV %. The transition to the “B” phase may not be clearly defined as the volumetric flow rate may change more slowly. The “B” phase contributes a greater than normal volume to the ventricular filling affecting cardiac functional reserve. This representation is useful for comparison with Normal Diastolic Function, Normal Diastolic Function with Hypovolemia, Mild Diastolic Dysfunction with Hypovolemia and Normal Compliance, Moderate Diastolic Dysfunction with Hypovolemia and Normal Compliance, Severe Diastolic Dysfunction, and Severe Diastolic Dysfunction with Hypovolemia.

By way of further example, the teachings of the present invention are useful in dealing with other than dysfunctional hearts, such as for an Anaerobic-Trained Athlete. Athletic training is a science of performance. For comparison to an Aerobic-Trained Athlete curve, the heart rate, EF, SV, and the total ventricular volume (VEDv) have been held constant. The IFV and IFV % are equivalent. Volume curve analysis can provide a great insight into cardiac functional capacity's ability to support the hemodynamic requirements of a given sport. By way of yet further example, reference is made to FIG. 17 illustrating a ventricular volume curve for an aerobically trained heart in a resting state and a calculation technique for one determination of R1 and R2, by way of example. FIG. 18 is the ventricular volume curve of FIG. 17 illustrating selected feature values, by way of example. FIG. 19 is a ventricular volume curve for an anaerobically trained heart in a resting state and FIG. 20 the ventricular volume curve of FIG. 19 illustrating the selected feature values. As above described for evaluating hearts having normal and dysfunctional diastolic conditions, the teachings of the present invention provides a useful evaluation for health hearts which may be compared to each other or to other than health hearts, as illustrated with reference to FIGS. 21-26 illustrating, by way of example, in table form and as a plot for various volumetric features observed for a normal heart, aerobic and anaerobic trained hearts, and hearts having mild, moderate and severe diastolic dysfunction, wherein the diastolic index is R1/R2. By way of yet further example, a volume curve of an anaerobic or heavy weight/low repetition training regimen consistent with sprinting or weight lifting may be observed and a comparison made with the Normal Diastolic Function or Aerobic Trained Athlete curves, or as desired using the Moro index (MI) as above described.

By way of example for comparison to Anaerobic curves for the heart rate, EF, SV, and the VEDv may be held constant. The IFV and IFV % are equivalent. Volume curve analysis can provide a great insight into cardiac functional capacity's ability to support the hemodynamic requirements of a given sport. This illustration displays a volume curve of an Aerobic or low weight/high repetition training regimen consistent with endurance sports. Comparisons may be made to the Normal Diastolic Function or Anaerobic-Trained Athlete.

There are many influences on cardiac volumetric performance that can dramatically affect filling dynamics as demonstrated with the various weighting techniques and variables. Changes in the diastolic filling period and the relationship to the initial filling time as seen with ventricular relaxation delays are presented with the indexes of DFP and DV %. Changes in SV can be seen in ventricles with systolic heart failure or severe valvular insufficiency affecting SV indexed measurements. Changes in heart rates affect can be presented with the T index. The effect of changes to the initial filling volume and the percent of SV are displayed with the V and % indexes, respectively.

An example of a normal volume curve relationships is displayed. In this example during early diastole (A) approximately 80% of the ventricle filling occurs as represented by the IFV % of 79.5. The diastasis phase (B) demonstrates little volume change in this Normal Resting Volume Curve. The majority of the remaining volume results from the atrial contraction (P).

The Moro Index R1 calculation of this curve is demonstrated resulting in a slope rate of 0.280 ml/msec. The R2 calculation is demonstrated as 0.007 ml/msec. The R1/R2 iteration of the Moro Index is 41.300, with the R2/R1 iteration of 0.024.

The sensitivity is displayed throughout the descriptors when even small changes affecting atrial preload or ventricular relaxation rates dramatically impact the Index. The range displayed in the Normal Comparison table for the examples given with the MI relationship of R1/R2 is from 11.227 to 41.3 when hypovolemia states are excluded. Hypovolemia causes a shift in the filling dynamics that mimics a more serious form of diastolic dysfunction as evidenced with the extremely low MI resulting.

An example of the mild diastolic dysfunction volume curve relationships is displayed. In this example during early diastole (A) approximately 67% of the ventricle filling occurs as represented by the IFV %. The diastasis phase (B) demonstrates a greater volume change when compared to the Normal Resting Volume Curve. The remaining volume results from the atrial contraction (P).

The Moro Index R1 calculation of this curve is demonstrated resulting in a slope rate of 0.230 ml/msec. The R2 calculation is demonstrated as 0.033 ml/msec. The R1/R2 iteration of the Moro Index is 7.053, with the R2/R1 iteration of 0.143.

The sensitivity is displayed throughout the descriptors when even small changes affecting atrial preload or ventricular relaxation rates dramatically impact the Index. The range displayed in the Normal Comparison table for the examples given with the MI relationship of R1/R2 is from 3.478 to 12.917 when hypovolemia states are excluded. Hypovolemia causes a shift in the filling dynamics that mimics a more serious form of diastolic dysfunction as evidenced with the extremely low MI resulting.

An example of the moderate diastolic dysfunction volume curve relationships is displayed. In this example during early diastole (A) approximately 33% of the ventricle filling occurs as represented by the IFV %. The diastasis phase (B) demonstrates a greater volume change when compared to the Mild Diastolic Dysfunction Resting Volume Curve. The remaining volume results from the atrial contraction (P).

The diastolic index R1 portion calculation of this curve is demonstrated resulting in a slope rate of 0.188 ml/msec. The R2 calculation is demonstrated as 0.090 ml/msec. The R1/R2 iteration of the index is 2.079, with the R2/R1 iteration of 0.479.

The sensitivity is displayed throughout the descriptors when even small changes affecting atrial preload or ventricular relaxation rates dramatically impact the Index. The range displayed in the Normal Comparison table for the examples given with the MI relationship of R1/R2 is from 1.775 to 4.743 when hypovolemia states are excluded. Hypovolemia causes a shift in the filling dynamics that mimics a more serious form of diastolic dysfunction as evidenced with the extremely low MI resulting.

An example of the severe diastolic dysfunction volume curve relationships is displayed. In this example during early diastole (A) approximately 14% of the ventricle filling occurs as represented by the IFV %. The diastasis phase (B) demonstrates a greater volume change when compared to the Moderate Diastolic Dysfunction Resting Volume Curve. The remaining volume results from the atrial contraction (P).

The diastolic index R1 portion calculation of this curve is demonstrated resulting in a slope rate of 0.124 ml/msec. The R2 calculation is demonstrated as 0.095 ml/msec. The R1/R2 iteration of the Moro Index is 1.302, with the R2/R1 iteration of 0.766. The sensitivity is displayed throughout the descriptors when even small changes affecting atrial preload or ventricular relaxation rates dramatically impact the Index. The range displayed in the Normal Comparison table for the examples given with the MI relationship of R1/R2 is from 1.000 to 1.86.

Two examples of athletic hearts demonstrating curves associated with different training focuses are presented for comparison to each other and the Normal Volume Curve to illustrate one of the possible uses of the MI in recognizing and possibly guiding training programs based on desired results. In these examples during early diastole (A) approximately 80% of the ventricle filling occurs as represented by the IFV % of 80 and 82%. The diastasis phase (B) demonstrates little volume change. The majority of the remaining volume results from the atrial contraction (P). The major difference is in the significant effect of IFT on the R1. The diastolic index R1 portion calculation of Aerobic curve is demonstrated resulting in a slope rate of 0.375 ml/msec. The R2 calculation is demonstrated as 0.009 ml/msec. The R1/R2 iteration of the Moro Index is 40.500, with the R2/R1 iteration of 0.024. The diastolic index R1 portion calculation of Anaerobic curve is demonstrated resulting in a slope rate of 0.288 ml/msec. The R2 calculation is demonstrated as 0.015 ml/msec. The R1/R2 iteration of the Moro Index is 19.327, with the R2/R1 iteration of 0.052. The sensitivity is displayed between the descriptors when even small changes affecting ventricular relaxation rates dramatically impact the Index. The comparison between the waveforms and resulting measurements and calculations demonstrates the sensitivity of the measure in detecting diastolic filling abnormalities for training and conditioning.

In one embodiment, the diastolic filling performance value can be used to manipulate the relaxation timing in a pacemaker. As discussed above, it has been common practice to use only the initial slope and initial peak velocity. In the present invention, however, it is illustrated that the change in slope and filling velocity between the initial and intermediate phases also provides an important indicator of diastolic performance. Broadly, the system and methods herein presented by way of example can be used to evaluate global right and left ventricular diastolic function. Such an evaluation can inform diagnosis and treatment strategies for diastolic heart failure across multiple imaging modalities. By way of non-limiting example, and as illustrated with reference to FIG. 27, the above described methods may be provided by a system 10 that employs cardiac imaging using cardiac magnetic resonance (CMR), cardiac computed tomography (CCT), nuclear cardiac imaging, echocardiography or speckle tracking technology, or any ventricular volume rendering technology for an imaging device 12 operable on a subject 14 in developing volume curves, such as those above described, wherein image data is transmitted to a processor 16 for quantifying the volumetric features and indices saved in storage 18 or provided in a display 20. As will come to the mind of those of ordinary skill in the art, now having the benefit of the teachings of the present invention, control parameters or preselected conditions may be input 22 for the process and the subject 14 may receive a stimulus 24, such as hydration, for making the above described comparisons and evaluating the diastolic function for the heart of the subject.

Although the invention has been described relative to various selected embodiments herein presented by way of example, there are numerous variations and modifications that will be readily apparent to those skilled in the art in light of the above teachings. It is therefore to be understood that, within the scope of the claims hereto attached and supported by this specification, the invention may be practiced other than as specifically described.

That which is claimed is:

1. A method for evaluating cardiac diastolic function, the method comprising:
measuring a volumetric flow of blood through a first heart;
determining a first volume change rate during a diastolic flow period of the volumetric flow;
determining a second volume change rate during the diastolic flow period, the second volume change rate following the first volume change rate;
formulating a first diastolic index from a combination of the first and second volume change rates for the first heart;
providing a second diastolic index for a second heart, wherein the second diastolic index is determined from diastolic volume change rates of the second heart; and
comparing the first diastolic index of the first heart with the second diastolic index of the second heart for evaluating the cardiac function of the first heart.

2. The method according to claim 1, wherein the comparing comprises selecting a condition of the second heart from a group of heart conditions including a normal diastolic function, a mild diastolic dysfunction, a moderate diastolic dysfunction, and a sever diastolic dysfunction, and the comparing the first diastolic index of the first heart with the second diastolic index thereof.

3. The method according to claim 2, wherein the comparing comprises comparing the first diastolic index to the second diastolic index for at least one of the heart conditions, and wherein the state is selected from a group consisting of resting, increased heart rate, decreased heart rate, increased heart rate and increased ejection fraction, increase heart rate and decreased ejection fraction, increased ejection fraction, hypovolemia, and hypovolemia and increased heart rate.

4. The method according to claim 3, further comprising weighting at least one volumetric flow feature of the first heart with the diastolic index, wherein the comparing comprises comparing the first weighted diastolic index of the first heart with the second weighted diastolic index of the second heart for evaluating the cardiac function of the first heart.

5. The method according to claim 4, wherein the at least one volumetric flow feature comprises at least one of a diastolic filling period (DFP) defined as a difference between a cycle duration and systolic duration, a stroke volume (SV), a cardiac cycle duration (T), an initial filling volume (V), an ejection fraction (EF) of a ventricle, a diastolic filling period (D), an initial filling volume percent (%), and a percentage (DV %) of initial filling time of a total diastolic filling period.

6. The method according to claim 5, wherein providing the initial filling volume percent (IFV %) comprises:
determining an initial filling volume (IFV) at a time where an intersection of extrapolations of the first and second straight line representations occurs; and
establishing the initial filling volume percentage (IFV %) from a relationship between the initial filling volume (IFV) to the stroke volume (SV) representative of the total filling volume.

7. The method according to claim 1, wherein the first volume change rate determining comprises:
establishing a first straight line representation of an initial filling volume portion of the diastolic flow period; and
designating an initial filling rate (R1) from the first straight representation, wherein the second volume change rate determining comprises establishing a second straight line representation of an intermediate filling volume portion; and
designating an intermediate filling rate (R2) from the second straight line representation, and
wherein the formulating of the diastolic index from a combination of the first and second volume change rates comprises providing the diastolic index as at least one of (R1)/(R2), (R2)/(R1) and (R1)×(R2).

8. The method according to claim 7, further comprising weighting at least one volumetric flow feature of the first heart with the diastolic index to provide a measure of diastolic filling performance, wherein the comparing comprises comparing the first weighted diastolic index of the first heart with the second weighted diastolic index of the second heart for evaluating the cardiac function of the first heart.

9. The method according to claim 8, wherein providing the diastolic filling performance comprises providing the weighted volumetric flow feature as a performance index, MI_{feature}, formed by at least one of:
MI_{feature}=volumetric flow feature×(R1/R2),
MI_{feature}=volumetric flow feature×(R2/R1),
MI_{feature}=(R1/R2)/volumetric flow feature,
MI_{feature}=(R2/R1)/volumetric flow feature,
MI_{feature}=volumetric flow feature/(R1/R2),
MI_{feature}=volumetric flow feature/(R2/R1),
MI_{feature}=volumetric flow feature×(R1×R2), and
MI_{feature}=volumetric flow feature/(R1×R2).

10. The method according to claim 9, wherein the cardiac function evaluating comprises evaluating at least one of volume in heart failure patients, cardiac diastolic performance, medication/titration for diastolic performance, an athletic training program, and cardiac reserve.

11. The method according to claim 10, wherein the athletic evaluating comprises prescribing an aerobic routine for a person when R1 is approximately equal to or less than R2×10.

12. A method for evaluating cardiac diastolic function, the method comprising:
determining a first volume change rate during an initial diastolic flow period of a volumetric flow in a cardiac function;
determining a second volume change rate during an intermediate diastolic flow period following the initial diastolic flow period;
formulating a diastolic index from a combination of the first and second volume change rates; and
comparing the diastolic index for each of a plurality of hearts.

13. The method according to claim 12, wherein the comparing comprises selecting a condition for at least one of the plurality of hearts from a group of heart conditions including a normal diastolic function, a mild diastolic dysfunction, a moderate diastolic dysfunction, and a sever diastolic dysfunction, and comparing the diastolic index for at least one of the plurality of hearts with the diastolic index of the selected heart having the condition.

14. The method according to claim 12, wherein the first volume change rate is represented by a first linear representation designated by R1, wherein the second volume change rate is represented by a second linear representation designated by R2, and wherein the diastolic index is a combination thereof.

15. The method according to claim 14, wherein the combination comprises at least one of R1/R2, R2/R1, and R1×R2.

16. The method according to claim 12, further comprising weighting the diastolic index with a volumetric flow feature for evaluation thereof.

17. The method according to claim 16, wherein the weighting comprises providing a performance index, MI_{feature}, including at least one of:
MI_{feature}=volumetric flow feature×(R1/R2),
MI_{feature}=volumetric flow feature×(R2/R1),
MI_{feature}=(R1/R2)/volumetric flow feature,
MI_{feature}=(R2/R1)/volumetric flow feature,
MI_{feature}=volumetric flow feature/(R1/R2),
MI_{feature}=volumetric flow feature/(R2/R1),
MI_{feature}=volumetric flow feature×(R1×R2), and
MI_{feature}=volumetric flow feature/(R1×R2).

18. The method according to claim 16, wherein the volumetric flow feature is selected from a group consisting of a diastolic filling period (DFP) defined as a difference between a cycle duration and systolic duration, a stroke volume (SV), a cardiac cycle duration (T), an initial filling volume (V), an ejection fraction (EF) of a ventricle, a diastolic filling period (D), an initial filling volume percent (%), and a percentage (DV %) of initial filling time of a total diastolic filling period.

19. The method according to claim 12, further comprising measuring the volumetric flow of blood through the heart, wherein the measuring comprises cardiac imaging from at least one of magnetic resonance, computed tomography, nuclear cardiac imaging, echocardiogram, and speckle tracking, and a volume rendering device.

20. A method for evaluating cardiac diastolic function, the method comprising:
measuring a first volumetric flow of blood through a first heart, wherein the first heart includes a first predetermined condition;
determining initial and intermediate volume change rates during a diastolic flow period of the first volumetric flow, the intermediate volume change rate following the initial volume change rate;
formulating a first diastolic index from a combination of the initial and intermediate volume change rates of the first volumetric flow;
quantifying at least one preselected feature of the first volumetric flow;
weighting the at least one volumetric flow feature with the diastolic index;
measuring a second volumetric flow of blood through a second heart, wherein the second heart includes a second predetermined condition;
determining initial and intermediate volume change rates during a diastolic flow period of the second volumetric flow, the intermediate volume change rate following the initial volume change rate;
formulating a second diastolic index from a combination of the initial and intermediate volume change rates of the second volumetric flow;
quantifying the at least one preselected feature selected for the first volumetric flow for the second volumetric flow;
weighting the at least one volumetric flow feature of the second volumetric flow with the second diastolic index; and
comparing the at least one weighted feature between each of the first and second volumetric flows.

21. The method according to claim 20, wherein the diastolic index forming comprises at least one of dividing the initial volume change rate by the intermediate volume change rate, dividing the intermediate volume change rate by the initial volume change rate, and multiplying the initial volume change rate by the intermediate volume change rate.

22. The method according to claim 20, wherein the at least one preselected feature comprises at least one of a diastolic filling period (DFP) defined as a difference between a cycle duration and systolic duration, a stroke volume (SV), a cardiac cycle duration (T), an initial filling volume (V), an ejection fraction (EF) of a ventricle, a diastolic filling period (D), an initial filling volume percent (V %), and a percentage (DV %) of the initial filling time of a total diastolic filling period.

23. The method according to claim 20, wherein the predetermined conditions of the hearts are selected from the group consisting of normal diastolic function, mild diastolic dysfunction, moderate diastolic dysfunction, and severe diastolic dysfunction.

24. The method according to claim 20, wherein the volumetric flow of blood measuring steps comprise measuring for at least one of the predetermined conditions of the heart functioning at rest, increased heart rate, decreased heart rate, increased volume and increased ejection fraction, increased volume and decreased ejection fraction, increased ejection fraction, hypovolemia, and hypovolemia and increased heart rate.

25. The method according to claim 20, further comprising;
comparing at least one of the predetermined conditions of the heart functioning at rest, increased heart rate, decreased heart rate, increased volume and increased ejection fraction, increased volume and decreased ejection fraction, increased ejection fraction, hypovolemia, and hypovolemia and increased heart rate for at least one of the heart having the normal diastolic function, the mild diastolic dysfunction, the moderate diastolic dysfunction, and the severe diastolic dysfunction.

26. The method according to claim 20, wherein the comparing step comprises selecting at least one distinguishable feature for the at least one predetermined condition, and wherein the at least one distinguishable feature illustrates a sensitivity sufficient for monitoring changes thereto for modification made therefor.
